(19) United States
(12) Patent Application Publication
(10) Pub. No.: US 2024/0099864 A1
(43) Pub. Date: Mar. 28, 2024

(54) ADJUSTABLE PROSTHETIC LIMB SOCKETS AND METHODS OF MAKING AND USING

(71) Applicant: Medical Creations, Inc., Aspen, CO (US)

(72) Inventor: Mark Joseph, Aspen, CO (US)

(21) Appl. No.: 18/374,624

(22) Filed: Sep. 28, 2023

Related U.S. Application Data
(60) Provisional application No. 63/410,900, filed on Sep. 28, 2022.

(51) Int. Cl. A61F 2/80; A61F 2/50

(52) U.S. Cl. CPC A61F 2/80; A61F 2/5046

(57) ABSTRACT

An adjustable prosthetic socket having adjustable features. For example, the socket can be heat moldable with one or more adjustable panels or members in communication with at least one tightening mechanism configured to reduce or adjust an inner circumference of the socket.

### CROSS-REFERENCE TO RELATED APPLICATIONS

This application claims benefit and priority under 35 U.S.C. § 119(e) to Provisional Patent Application Ser. No. 63/410,900 filed Sep. 28, 2022, which is hereby incorporated by reference in its entirety as if fully set forth herein.

### BACKGROUND OF THE INVENTION

### Field of the Invention

The present disclosure generally relates to medical devices, adjustable prosthetic sockets for prosthetic limbs, and more specifically to a heat moldable adjustable prosthetic socket having improved conformability to a patient's residual limb and allowing a user to make rapid adjustments.

### SUMMARY OF THE INVENTION

The present disclosure describes an adjustable prosthetic socket also referred to herein as an adjustable prosthetic limb socket for an amputated limb that is formed directly to the residual limb of the patient that becomes formable, pliable, and stretchable and is adjustable by an end user.

An advantage of the invention is to provide an adjustable socket configured to allow a user to readily and easily reduce or expand the internal volume of the prosthetic socket.

Additional features and advantages of the invention will be set forth in the description which follows and, in part, will be apparent from the description or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, in one embodiment, an adjustable prosthetic socket includes a pre-made socket including a proximal open end, a distal end that is substantially closed, and a receptacle extending from the distal end to the proximal open end. The pre-made socket is configured to at least partially surround and secure a residual limb. The adjustable prosthetic socket has at least one recess in a wall of the pre-made socket and at least one adjustable panel having a dimension arranged in the recess. The socket also has a tightening mechanism, which includes a base rotationally coupled to a dial where the tightening mechanism is coupled to a tensioning line and configured not to rotate more than 360 degrees.

In another aspect of the present invention, a heat moldable adjustable prosthetic socket includes a proximal open end, a distal end, and a wall extending from the distal end to the proximal open end. The heat moldable adjustable prosthetic socket is configured to at least partially surround and secure a residual limb and includes a recess in the wall, a panel arranged in the recess, and a tightening mechanism coupled to a tensioning line capable of changing an inner circumference of the heat moldable adjustable prosthetic socket by moving the panel from a first position to a second position, thereby reducing an inner circumference of the socket.

In yet another aspect of the invention, a method for making an adjustable prosthetic socket includes providing a flexible inner pre-socket, including a proximal end, a distal end, and a wall surrounding and extending from the distal end to the proximal end. The flexible inner pre-socket is a heat moldable socket; alternatively, a conventional liner may be utilized. Next, the method includes providing a substantially flat heat moldable adjustable panel and heating said panel to a shaping temperature. After heating, the panel is molded to conform to a curvature of an outer side wall of the flexible inner pre-socket at a predetermined position of the flexible inner pre-socket. The method also includes arranging the flexible inner pre-socket and formed panel in a receptacle of a heat moldable socket to form an assembly. Next, the assembly is heated to a shaping temperature and molded onto a residual limb or a model of a residual limb to form a recess in a sidewall of the heated heat moldable socket. A tightening mechanism is attached to the socket at a predetermined location. A tensioning line is connected to the tightening mechanism and the panel.

In still yet another aspect of the invention, a method for using an adjustable prosthetic socket includes changing an inner circumference of a heat molded prosthetic socket with a tensioning mechanism by moving an adjustable member arranged in a recess of the socket from a first position outwardly relative to an inner sidewall of the socket to a second position.

In yet another aspect of the invention, an adjustable prosthetic socket kit includes components of the adjustable socket and instructions for using the components to create the adjustable prosthetic socket. For example, the kit can include a tensioning line, an alignment template configured for use for forming one or more alignment holes in a heat moldable socket, a tightening mechanism configured to be coupled with a tensioning line, an adjustable member, and one or more of instructions for creating an adjustable prosthetic socket.

This Summary section is neither intended to be, nor should be, construed as being representative of the full extent and scope of the present disclosure. Additional benefits, features, and embodiments of the present disclosure are set forth in the attached figures, in the description herein, and as described by the claims. Accordingly, it should be understood that this Summary section may not contain all of the aspects and embodiments claimed herein.

Additionally, the disclosure herein is not meant to be limiting or restrictive in any manner. Moreover, the present disclosure is intended to provide an understanding to those of ordinary skill in the art of one or more representative embodiments supporting the claims. Thus, it is important that the claims be regarded as having a scope including constructions of various features of the present disclosure insofar as they do not depart from the scope of the methods and apparatuses consistent with the present disclosure (including the originally filed claims). Moreover, the present disclosure is intended to encompass and include obvious improvements and modifications of the present disclosure.

### BRIEF DESCRIPTION OF THE DRAWINGS

The accompanying drawings, which are included to provide a further understanding of the invention and are incorporated in and constitute a part of this specification, illustrate embodiments of the invention and, together with the description, serve to explain the principles of the invention.

In the drawings:

FIG. 1 illustrates a right backside perspective view of an adjustable prosthetic socket in accordance with an embodiment of the invention;

FIG. 2 illustrates a left backside perspective view of the adjustable prosthetic socket of FIG. 1;

FIG. 3 illustrates a top down view of the adjustable prosthetic socket of FIG. 1 having an adjustable panel or member in a first orientation;

FIG. 4 illustrates a top down view of the adjustable prosthetic socket of FIG. 1 having an adjustable panel or member in a second orientation;

FIG. 5 illustrates an adjustable panel or member of the adjustable prosthetic socket of FIG. 1;

FIG. 6 illustrates a magnified view of an actuator of the adjustable prosthetic socket of FIG. 1;

FIG. 7 illustrates a disassembled or exploded view of the actuator of the adjustable prosthetic socket of FIG. 1;

FIG. 8 illustrates a magnified view of the actuator of the adjustable prosthetic socket of FIG. 1 in a first orientation;

FIG. 9 illustrates a magnified view of the actuator of the adjustable prosthetic socket of FIG. 1 in a second orientation;

FIGS. 10-16 collectively illustrate a method of making an adjustable prosthetic socket in accordance with another embodiment of the invention;

FIG. 17 illustrates a left backside perspective view of an adjustable prosthetic socket in accordance with another embodiment of the invention; and

FIG. 18 illustrates a right backside perspective view of an adjustable prosthetic socket in accordance with another embodiment of the invention.

### DETAILED DESCRIPTION OF THE ILLUSTRATED EMBODIMENTS

For the purposes of promoting an understanding of the principles of the present disclosure, reference will now be made to the exemplary embodiments illustrated in the drawing(s), and specific language will be used to describe the same.

Appearances of the phrases an “embodiment,” an “example,” or similar language in this specification may, but do not necessarily, refer to the same embodiment, to different embodiments, or to one or more of the figures. The features, functions, and the like described herein are considered to be able to be combined in whole or in part one with another as the claims and/or art may direct, either directly or indirectly, implicitly or explicitly.

As used herein, “comprising,” “including,” “containing,” “is,” “are,” “characterized by,” and grammatical equivalents thereof are inclusive or open-ended terms that do not exclude additional unrecited elements or method steps unless explicitly stated otherwise.

Persons skilled in the art will readily appreciate that various aspects of the present disclosure can be realized by any number of methods and articles configured to perform the intended functions. Stated differently, other methods and articles can be incorporated herein to perform the intended functions. It should also be noted that the accompanying drawing figures referred to herein are not all drawn to scale but may be exaggerated to illustrate various aspects of the present disclosure, and in that regard, the drawing figures should not be construed as limiting. Finally, although the present disclosure may be described in connection with various principles and beliefs, the present disclosure should not be bound by theory.

In order to more fully appreciate the present disclosure and to provide additional related features, the following references are incorporated therein by reference for the following teachings and in their entirety:

(1) U.S. Pat. No. 8,443,501 by Mahon, which discloses prosthesis devices that can include sockets having adjustable features. In one example, a socket includes one or more panels that can move outwardly or inwardly relative to a receptacle portion of the socket. The panels can be moved by tightening a tensioning line.

(2) U.S. Pat. No. 9,956,094 by Mahon, which discloses prosthesis devices that can include sockets having adjustable features. In one example, a socket includes one or more panels that can move outwardly or inwardly relative to a receptacle portion of the socket. The panels can be moved by tightening a tensioning line.

(3) U.S. Pat. No. 10,004,614 by Johnson, which discloses a disarticulated compression socket configured to secure a residual limb. The disarticulated compression socket may include a rigid socket frame having one or more compression apertures each having one or more disarticulated compression inserts. Each disarticulated compression insert may be coupled with, and responsive to, a compression actuator configured to adjust the disarticulated compression insert individually, or in concert. In one preferred embodiment, at least one compression actuator may be coupled with one or a plurality of disarticulated compression inserts and further configured to retract and/or expand the coupled disarticulated compression inserts securing the residual limb within said socket frame. Control of the compression actuators may be manual or automatic in response to a signal from a sensor. Additional embodiments may also include one or more lateral release channels configured to accommodate soft tissue expansion of the residual limb as it is compressed and/or secured within the socket frame.

(4) U.S. Pat. No. 10,406,003 by Johnson, which discloses a disarticulated compression socket configured to secure a residual limb. The disarticulated compression socket may include a rigid socket frame having one or more compression apertures each having one or more disarticulated compression inserts. Each disarticulated compression insert may be coupled with, and responsive to, a compression actuator configured to adjust the disarticulated compression insert individually, or in concert. In one preferred embodiment, at least one compression actuator may be coupled with one or a plurality of disarticulated compression inserts and further configured to retract and/or expand the coupled disarticulated compression inserts securing the residual limb within said socket frame. Control of the compression actuators may be manual or automatic in response to a signal from a sensor. Additional embodiments may also include one or more lateral release channels configured to accommodate soft tissue expansion of the residual limb as it is compressed and/or secured within the socket frame.

(5) U.S. Pat. No. 10,918,502 by Mahon, which discloses prosthesis devices that can include sockets having adjustable features. In one example, a socket includes one or more panels that can move outwardly or inwardly relative to a receptacle portion of the socket. The panels can be moved by tightening a tensioning line.

(6) U.S. Pat. No. 11,083,602 by Mahon, which discloses a prosthesis device that can include sockets having adjustable features. In one example, a socket includes one or more panels that can move outwardly or inwardly relative to a receptacle portion of the socket. The panels can be moved by tightening a tensioning line.

In one embodiment, adjustable prosthetic limb sockets are used to secure prosthetic limbs to the residual limb of a patient. These sockets have adjustable features and can include one or more panels that can move outwardly or inwardly relative to an inner side wall of the socket. The panels can move with one or more tensioning lines and a tightening mechanism. In many cases, the prosthetist and patient select an appropriate liner to apply to the residual limb. The liner reduces discomfort (such as chafing or rubbing) between the skin of the residual limb and a socket. The liner also has a very high friction interior that adheres to the skin to hold it in place during movement and has a connection mechanism to the socket that can vary. The adjustable socket is applied over the liner and acts to support and suspend a prosthetic limb to the residual limb of the patient. Stated another way, the liner is positioned between a residual limb and a socket, and the actual prosthetic limb is coupled to the socket. Optionally and/or alternatively, the liner can be a flexible inner socket as described herein.

In one embodiment, an adjustable prosthetic socket includes a heat moldable socket having a proximal open end, a distal end that is substantially closed, and a wall surrounding and extending from the distal end to the proximal open end. The heat moldable socket is configured to at least partially surround and secure a residual limb and includes at least one recess in the wall of the heat moldable socket. The recess is dimensioned to receive at least one adjustable panel. The panel resides entirely inside the heat moldable socket and does not extend outside of the heat moldable socket during operation. The socket also includes a tightening mechanism coupled to a tensioning line, e.g., a cable or wire, configured to change an inner circumference of the heat moldable socket by operation of the tightening mechanism, thereby moving at least one adjustable panel from a first position to a second position in order reduce at least a portion of the inner circumference of the socket.

In one embodiment, an adjustable prosthetic socket includes a pre-made socket including a proximal open end, a substantially closed distal end that is a substantially closed end, and a receptacle extending from the distal end to the proximal open end. The pre-made socket is configured to at least partially surround and secure a residual limb. The adjustable prosthetic socket has at least one recess in a wall of the pre-made socket and at least one adjustable panel having a dimension arranged in the recess. The socket also has a tightening mechanism that includes a base rotationally coupled to a dial where the tightening mechanism is coupled to a tensioning line and configured not to rotate more than 360 degrees.

In one embodiment, a heat moldable adjustable prosthetic socket includes a proximal open end, a distal end, and a wall extending from the distal end to the proximal open end. The heat moldable adjustable prosthetic socket is configured to at least partially surround and secure a residual limb and includes a recess in the wall, a panel arranged in the recess, and a tightening mechanism coupled to a tensioning line capable of changing an inner circumference of the heat moldable adjustable prosthetic socket by moving the panel from a first position to a second position, thereby reducing an inner circumference of the socket.

In one embodiment, a method for making an adjustable prosthetic socket includes providing a flexible inner pre-socket including a proximal end, a distal end, and a wall surrounding and extending from the distal end to the proximal end. The flexible inner pre-socket is heat moldable; alternatively, a conventional liner may be utilized. Next, the method includes providing a substantially flat heat moldable adjustable panel and heating said panel to a shaping temperature. After heating, the panel is molded to conform to a curvature of an outer side wall of the flexible inner pre-socket at a predetermined position of the flexible inner pre-socket. The method also includes arranging the flexible inner pre-socket and formed panel in a receptacle of a heat moldable socket to form an assembly. Next, the assembly is heated to a shaping temperature and molded onto a residual limb or a model of a residual limb to form a recess in a sidewall of the heated heat moldable socket. A tightening mechanism is attached to the socket at a predetermined location. A tensioning line is connected to the tightening mechanism and the panel.

In one embodiment, a method for using an adjustable prosthetic socket includes changing an inner circumference of the socket with a tensioning mechanism by moving an adjustable member arranged in a recess of the socket from a first position outwardly relative to an inner sidewall of the socket to a second position.

In one embodiment, an adjustable prosthetic socket kit includes components of the adjustable socket and instructions for using the components to create the adjustable prosthetic socket. For example, the kit can include a tensioning line, an alignment template configured for use for forming one or more alignment holes in a heat moldable socket, a tightening mechanism capable of being coupled with a tensioning line, an adjustable member, and one or more of instructions for creating an adjustable prosthetic socket.

In one embodiment, the tightening mechanism includes a base and a knob. Portions of the tensioning line extend into the tightening mechanism. The tightening mechanism is configured to collect portions of the tensioning line within the tightening mechanism. In certain embodiments, rotation of the knob draws the tensioning line into the tightening mechanism so as to wrap the tensioning line about a spool member. For example, the tightening mechanism can operate in a manner such as a ratcheting device.

In one embodiment, the tensioning line can include more than one tensioning line and can include any suitable arrangement, such as, for example, a wire, cable, or the like, and may be relatively flexible or pliant in a transverse direction but relatively inextensible in a longitudinal direction. The tensioning line can include any suitable material, such as, for example, alloy, metal, polyester, polymer, fabric, combinations of the same and the like.

In one embodiment, the adjustable socket includes an adjustable upper portion and a fixed lower portion. In certain embodiments, the adjustable upper portion has a first pliability in a given temperature range, which is greater than the pliability of the lower portion in the same temperature range. In one embodiment, the lower portion serves to support the socket during heat forming yet is still conformable when heated. The lower portion also has the means to attach the prosthetic limb in an adjustable fashion and has an attachment member for various mechanisms to lock a gel liner to the socket. In this case, the lowest portion that performs this function is not heated, so it retains its shape and mechanical properties.

In one embodiment, an adjustable prosthetic socket includes a proximal insertion opening and an inner circumference, which at least partially surrounds a residual limb, and at least one adjustable region for a prosthesis component configured to reduce an inner circumference of the prosthetic socket. An actuator, when adjusted, changes the inner circumference of the prosthesis socket.

In one embodiment, the method includes controlling an adjustment of the inner circumference of the prosthetic socket.

In one embodiment, the method includes making an adjustable prosthetic socket.

In one embodiment, at least a portion of the prosthetic socket, e.g., conical cup, is heat moldable and formed as described in and with reference to U.S. Pat. No. 10,751,202, U.S. patent application Ser. Nos. 17/001,380 and 17/446,115, each of which is hereby incorporated by reference as if fully set forth herein. The adjustable portion of the socket is formed as described herein.

In one embodiment, the adjustable prosthetic socket is used for receiving at least a portion of a residual limb and is further configured to have a plate or other mechanism that is configured to reduce the internal circumference of the prosthetic socket.

In one embodiment, the adjustable prosthetic socket includes a proximal opening and an inner circumference configured to at least partially surround a residual limb. An inner wall of the prosthetic socket includes at least one recess for receiving a movable element, e.g., a panel. The prosthetic socket has no other opening or cutouts other than the proximal opening. The movable element is configured to move from one position to a second position by an actuator. The actuator is configured to rotate and pull one or more cables in communication with the movable element to change the prosthetic socket's inner circumference. In one embodiment, the socket is heat moldable as described herein and the movable element is also heat moldable.

In one embodiment, the panel is heat moldable and arranged between the socket and a liner or flexible inner socket. The flexible inner socket is described with reference to U.S. patent application Ser. Nos. 16/897,425 and 17/901,375, each of which is hereby incorporated by reference as if fully set forth herein. In this embodiment, the panel resides partially in a recess formed while making the socket, e.g., a sidewall.

Reference will now be made in detail to embodiments of the invention, examples of which are illustrated in the accompanying drawings and appendices.

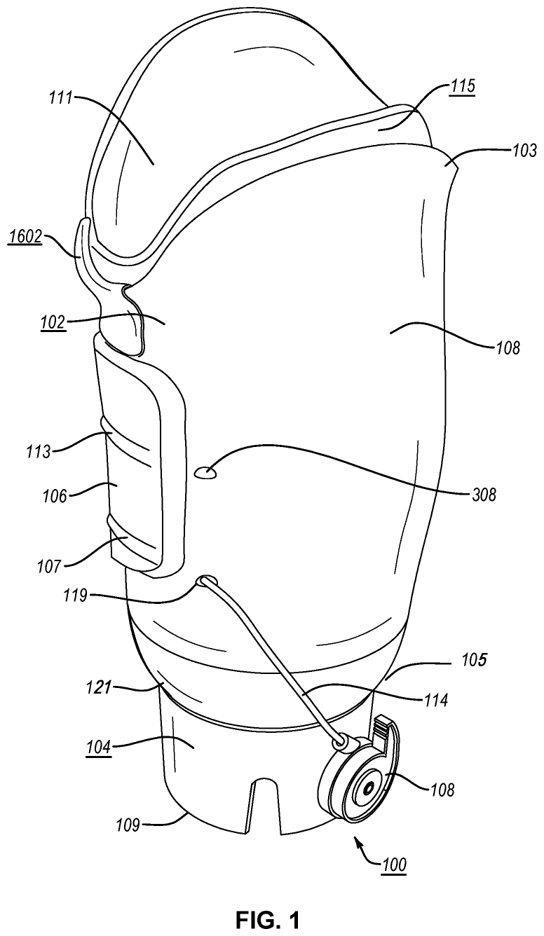

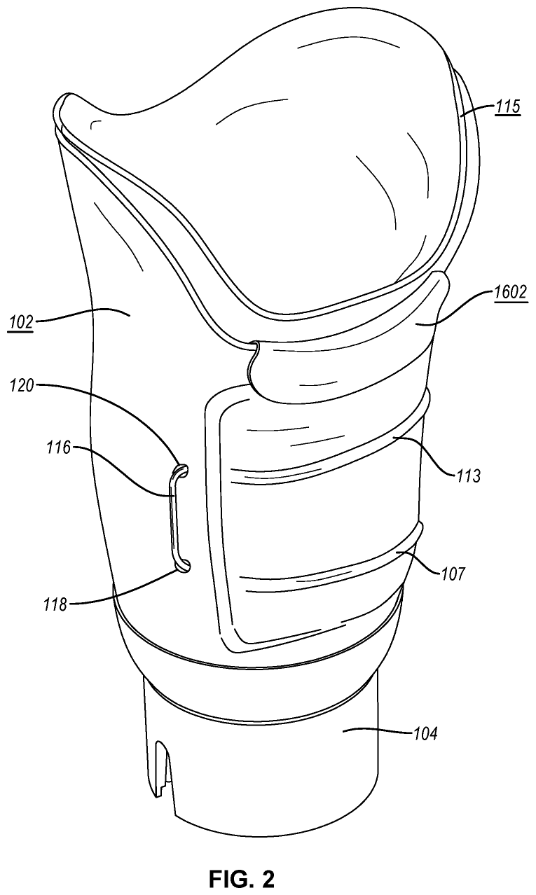

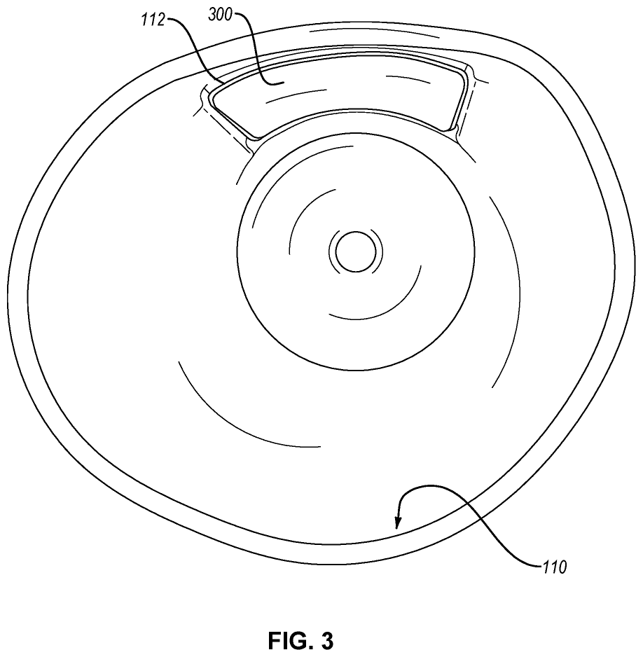

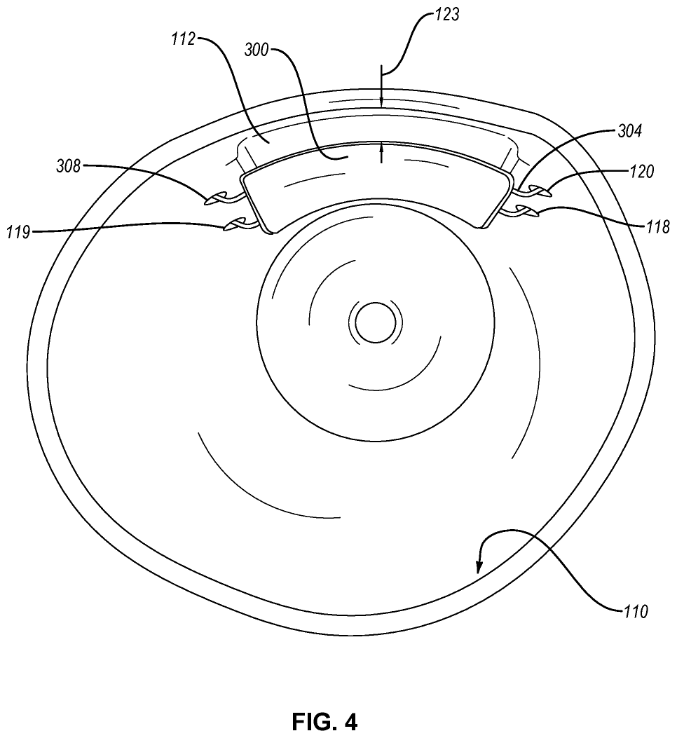

FIG. 1 illustrates a right backside perspective view of an adjustable prosthetic socket in accordance with an embodiment of the invention. FIG. 2 illustrates a left backside perspective view of the adjustable prosthetic socket of FIG. 1. FIG. 3 illustrates a top down view of the adjustable prosthetic socket of FIG. 1 having an adjustable panel or member in a first orientation. FIG. 4 illustrates a top down view of the adjustable prosthetic socket of FIG. 1 having an adjustable panel or member in a second orientation.

Referring to FIGS. 1-4, the adjustable prosthetic limb socket is generally depicted with reference to number 100. The adjustable prosthetic socket 100 is used to secure a residual limb. In a preferred embodiment, the adjustable prosthetic socket 100 includes a conical cup 102 constructed from a heat moldable material with a pliability for shaping time and other properties and is described with reference to U.S. Pat. No. 10,751,202, U.S. patent application Ser. Nos. 17/001,380 and 17/446,115, each of which is hereby incorporated by reference as if fully set forth herein. For example, the heat moldable materials have a shaping temperature in a range of about 120° F. to about 305° F. and any sub-range within. In one embodiment, the adjustable prosthetic socket 100 is a pre-made injection molded thermoplastic elastomer socket configured to be heated to a formable temperature so that the injection molded premade thermoplastic elastomer socket becomes formable and stretchable at a shaping temperature as described herein.

In this embodiment, the adjustable prosthetic limb socket 100 is configured to receive a lower portion of a residual limb, such that a portion of the residual limb can sit inside the conical cup 102 and be secured. In addition, as known in the art, a prosthetic mechanical lower leg is attached to a lower portion of the socket 100 to allow a person with a below or above the knee amputation to walk using the socket plus a prosthesis.

The adjustable prosthetic socket 100 includes a conical cup 102 and a lower portion 104. In this embodiment, a tightening mechanism 108, e.g., an actuator, is adjustably attached to the lower portion 104. The actuator is in mechanical communication with one or more adjustable panels 300 via one or more tensioning lines 304 and configured to be arranged inside the conical cup 102 and configured to move or adjust the panel from a first position to a second position. The adjustable panel or member 300 is configured to modify compression on the residual to allow a user to dynamically adjust the amount of inner circumference reduction at any given time.

In this embodiment, the conical cup 102 is in the shape of a hollow, deep, or elongated cup that is generally cylindrical in shape, having a first end 103 and a second end 105. The lower portion 104 includes a first end 121 and a second end 109. The lower portion 104 is configured to also include a cup region to receive a portion surrounding a portion of the conical cup 102. The conical cup 102 is in the shape of a hollow, deep, or elongated cup that is generally cylindrical, having a first end 103 and a second end 105. The first end 103 of the conical cup 102 is formed with an opening 111, via which the residual limb can be inserted into the conical cup 102 and supported.

In one embodiment, the conical cup 102 is shapeable or moldable after being heated at a shaping temperature. In one embodiment, after heating to a shapeable or moldable temperature, the conical cup is configured to be applied directly to a residual limb or to a model of the residual limb as described in U.S. Pat. No. 10,751,202, U.S. patent application Ser. Nos. 17/001,380 and 17/446,115, each of which is hereby incorporated by reference as if fully set forth herein. The conical cup can be made of a heat moldable, thermoformable material, e.g., a thermoplastic plastic, polymeric material, combinations of the same, and the like. The shaping temperature can be in the range from about 120° F. to about 305° F. and any sub-range within. The shaping time can be in the range of about 5 minutes to about 15 minutes or any sub-range within.

The adjustable panel or member 300 is also constructed from a heat moldable material with a pliability for shaping time and other properties as described with reference to U.S. Pat. No. 10,751,202, U.S. patent application Ser. Nos. 17/001,380 and 17/446,115, each of which is hereby incorporated by reference as if fully set forth herein. The panel can be constructed with materials including a heat moldable, thermoformable material, e.g., a thermoplastic plastic, polymeric material, combinations of the same, and the like. Optionally and/or alternatively, the panel can be constructed from a non-heat moldable plastic or polymer material with a preconfigured geometry. The panel can also be reinforced with carbon fiber or other reinforcement additives as known in the art.

In this embodiment, the conical cup 102 does not include one or more outer layers but can include an outer layer, e.g., a decorative layer, as described in detail with reference to U.S. patent application Ser. No. 17/446,115, which is hereby incorporated by reference as if fully set forth herein. After heating to the shaping temperature, the outer surface of the conical cup 102 can be a wavy, bumpy, and/or uneven surface. The outer layer corrects these issues and can add more strength along with cosmetic features.

In some embodiments, the lower portion 104 can be formed with blow molding, injection molding, rotational molding, or other techniques. In one embodiment, the lower portion 104 is formed with a material that is not moldable at a shaping temperature. The lower portion 104 includes a material more rigid than the conical cup portion 102 at a shaping temperature or room temperature. The lower portion includes a material such as acrylonitrile butadiene styrene (ABS), nylon, polycarbonate, or a fiberglass or carbon filled polymer. Optionally and/or alternatively, the lower portion 104 is made from the same material as the conical cup 102. In one embodiment, the lower portion 104 is not heated or is heated at a lower temperature than the shaping temperature when the conical cup 102 and the adjustable member 300 are heated, such as at a temperature below about 250° F. In one embodiment, the lower portion 104 and the conical cup 102 can be one piece and formed as a single piece. Optionally and/or alternatively, the lower portion and conical cup are formed as described with reference to U.S. Pat. No. 10,751,202, U.S. patent application Ser. Nos. 17/001,380 and 17/446,115, each of which is hereby incorporated by reference as if fully set forth herein.

Optionally and/or alternatively, the adjustable socket can also utilize a flexible inner socket 115, which can be made using a preformed flexible inner socket (also referred to as flexible inner pre-socket) as described in detail with reference to U.S. patent application Ser. Nos. 16/897,425 and 17/901,375, each of which is hereby incorporated by reference as if fully set forth herein. In addition, other conventional liners may also be utilized. In addition, conventional liners may also be used.

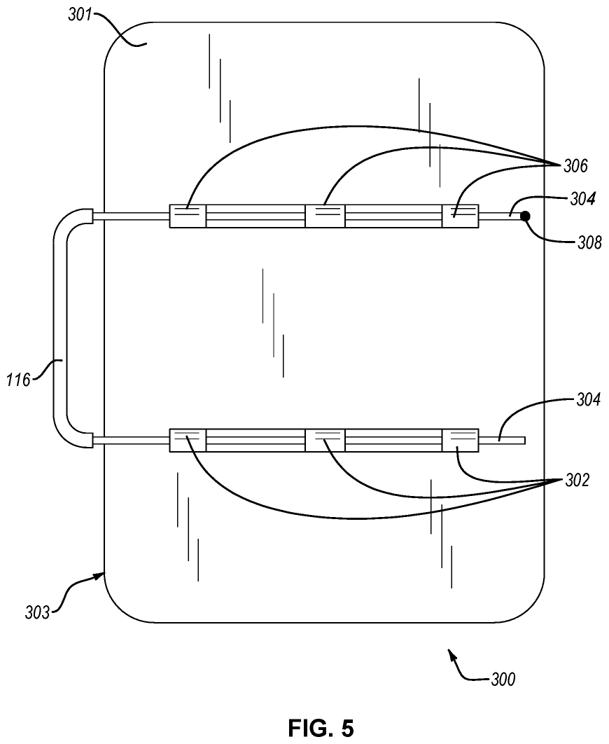

Referring again to FIGS. 1-5, conical cup 102 includes a recess portion 112 in a sidewall of the conical cup 102 and recess or recess portion 112 that is sized to receive at least a portion of the adjustable member 300. In a preferred embodiment, the recess 112 is sized such that when the adjustable member 300 is in a resting position (not expanded), the inner circumference of the inner sidewall 110 is substantially as it would have been without the adjustable member 300 or recess 112. The recess 112 can have a depth substantially equal to the dimensions such as the thickness, length, and height of the adjustable member 300, or greater than the dimensions of the adjustable member 300, or less than the dimensions of the adjustable member 300, or any combination of the same. The outer sidewall 108 of the conical cup has a bulge portion or protruded region 106 opposite the recess 112. The outer sidewall 108 of the conical cup has no opening, void, or cutout region through a sidewall of the conical cup for recess or adjustable member 300. That is, the adjustable member 300 resides inside the conical cup 102 and never outside the conical cup 102. It is believed that the lack of opening, void, or cutout region in the sidewall enhances the overall strength of the socket and performance of the adjustable member 300.

The inner sidewall 110 also includes channels or partial channels 107 and 113 configured to receive one or more cables, wires, or other linkage from an actuator 108. In this embodiment, the wire or wires 304 are coupled to the actuator 108, which is configured to rotate less than 360 degrees to move the wire or wires 304. The outer sidewall 108 shows channels 107 and 113 as bulges or protrusions.

Optionally and/or alternatively, the adjustable prosthetic socket 100 also includes a first guide tube 114 extending from the actuator 108 to an input 119 of the first channel 107 and a second tube 116 extending from output 118 of the first channel 107 to an input 120 of the second channel 113. The first guide tube 114 and second guide tube 116 can be a flexible or rigid tube, or “J” tube, for guiding the wire. The guide tube can be constructed of one or more of the following materials, including a metal material, an alloy material, a plastic material, a polymer material, combinations of the same, and the like.

Referring to FIGS. 1-5, the adjustable member or panel 300 includes one or more alignment sections 302 configured to receive, guide, and hold at least a portion of one or more tensioning lines 304. One or more tensioning lines can include a wire, a cable, a lace, a woven material, a braided material, a non-braided material, combinations of the same, and the like. Each of the tensioning lines can be formed by one or more of a metal material, a polyester material, an alloy material, a synthetic material, combinations of the same and the like. In this embodiment, a wire or cable is utilized. The wire 304 extends from an actuator 108 through a first guide tube 114, an input 116, and channel or partial channel 107 across a backside 301 of the panel 300. The wire 304 further extends through one or more alignment sections 304 out of channel 107, output 118 through the second guide tube 116, and then into the input 120 and channel or partial channel 113. Wire 304 extends through one or more second alignment sections 306 out of the channel or partial channel to a termination point 308. In this embodiment, the termination point 308 can include any termination to secure the end of the wire 304 to at least a portion of the conical cup 102, e.g., a knot, a nut, and the like. One or more alignment sections 304 and 306 are also configured to help with maintaining channels for the wire 304 when formed in the recess, e.g., shown in FIG. 4. Optionally, and/or alternatively, the adjustable panel 300 is made by three-dimensional (3D) printing, injection molding, blow molding, combinations of the same and the like into the desired geometric configuration.

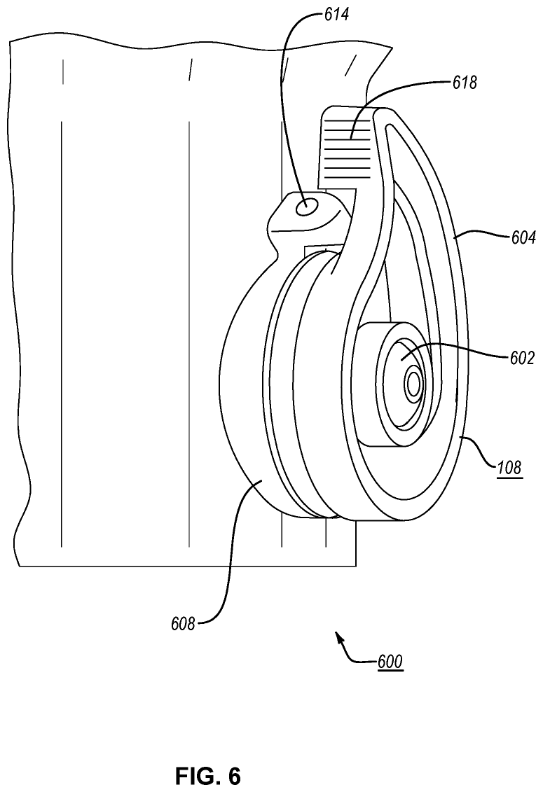

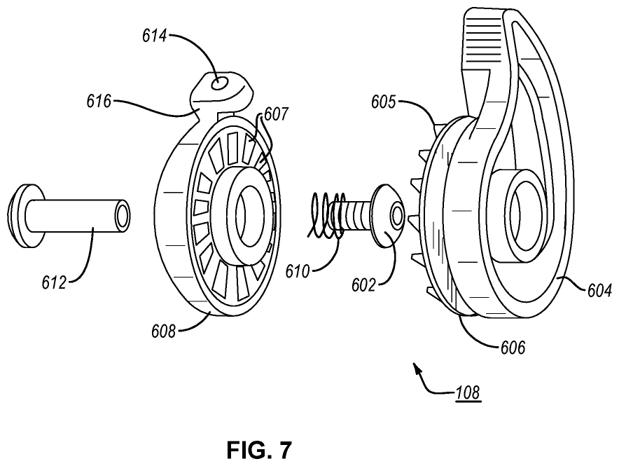

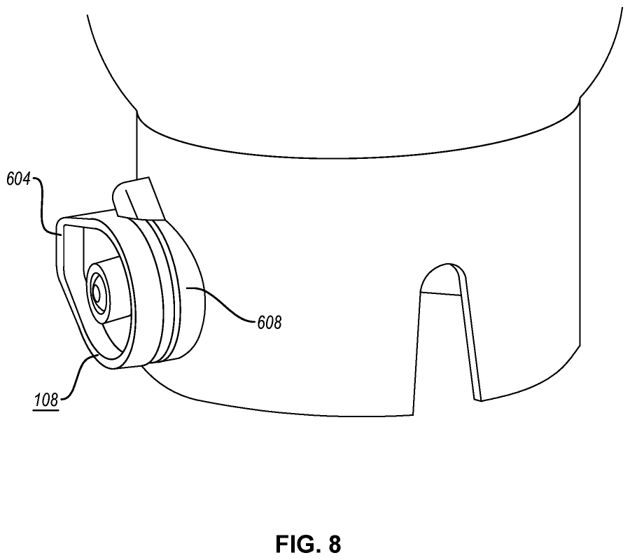

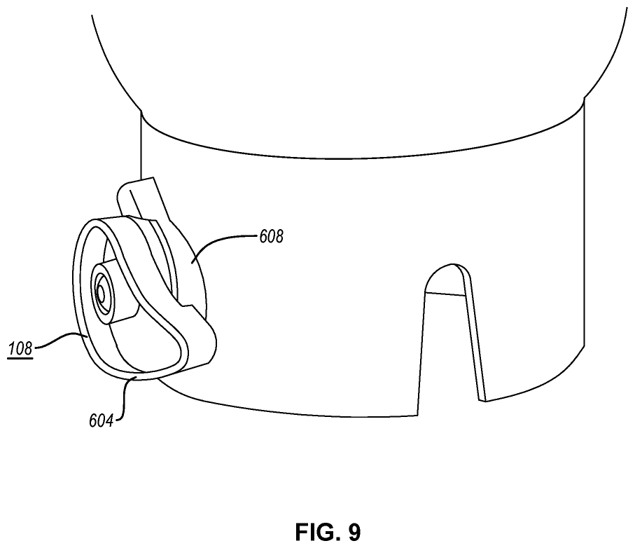

FIG. 6 illustrates a magnified view of an actuator of the adjustable prosthetic socket of FIG. 1. FIG. 7 illustrates a disassembled or exploded view of the actuator of the adjustable prosthetic socket of FIG. 1. FIG. 8 illustrates a magnified view of the actuator of the adjustable prosthetic socket of FIG. 1 in a first orientation. FIG. 9 illustrates a magnified view of the actuator of the adjustable prosthetic socket of FIG. 1 in a first orientation.

Referring to FIGS. 6-9, the actuator 108 is configured to move the adjustable panel 300 from a first position to a second position by adding tension to the wire 304 or decreasing tension on the wire 304. In this embodiment, the actuator cannot rotate more than one of 360 degrees, 180 degrees, and 90 degrees. The actuator 108, in this embodiment, is also configured to move from a plurality of preset positions in a clicking fashion. After moving, the actuator is substantially locked in the preset position. That is, the actuator 108 is configured to rotate through a first position to a second position to tension the wire 304. The actuator 108 includes a handle 604 configured to rotate to tension a wire when rotated in a first direction and release or reduce tension on the wire when rotated in a second direction. The actuator 108 engages a disk 606 with a plurality of gears 605 protruding from the disk at a predetermined spacing. The predetermined spacing may be uniform or not uniform and is spaced to size the amount of tension on each movement. The plurality of gears 605 are configured to engage a sprocket device 608 with a plurality of engagement holes 607 at the same predetermined spacing as gears 605. The plurality of engagement holes 607 are configured to at least partially receive the plurality of gears 605. When the plurality of gears 605 are engaged in the plurality of engagement holes 607, the actuator is fixed in a locked position.

The actuator 108 also includes an attachment member 602, including a biasing member 610. The attachment member 602 attaches the handle 604 and disk 605 to the sprocket 608 in a biased configuration. The biasing member 610 can include a spring or other biasing member as known in the art. The biasing member 610 forces the disk 606 and plurality of gears 605 in a locked position in the engagement 607. Due to the shape of the plurality of gears 605 and engagement holes 607, the biasing member can be partially released by rotating the handle 604 from a first position to a second position. That is, the geometry of the plurality of gears 605 and engagement holes 607 has a slope or ramp that allows the biasing member to be forced into an unengaged state allowing the gears to move from a first engagement hole to a second engagement hole, etc. and allows for rotation to add tension to the wire or reduce tension on the wire.

The attachment member 612 is configured to receive the attachment member 602 and secure the handle 604, disk 605, and sprocket 608 together in a biased configuration. The actuator 108 can be attached to the lower portion 104 or the conical cup 102 with an attachment mechanism (not shown), e.g., screws, rivets, glue, etc. A wire (not shown) enters input 614 and is attached to disk 605, and the wire at least partially wraps around the sprocket member 608.

In operation, handle 604 of the attachment member 108 is configured to rotate less than 360 degrees, as shown in FIGS. 8 and 9. The attachment member in FIG. 8 shows a first position corresponding to the position of the adjustable member 300 in FIG. 3, where there is no reduction in circumference or compressive force. In this position, the adjustable member 300 resides mainly in recess 112. The attachment member in FIG. 9 shows a second position corresponding to the position of the adjustable member 300 in FIG. 3, which is tensioned. In this position, the adjustable member 300 resides removed from recess 112, thereby adding a compression force to the residual limb and reducing the interior circumference of the socket 102.

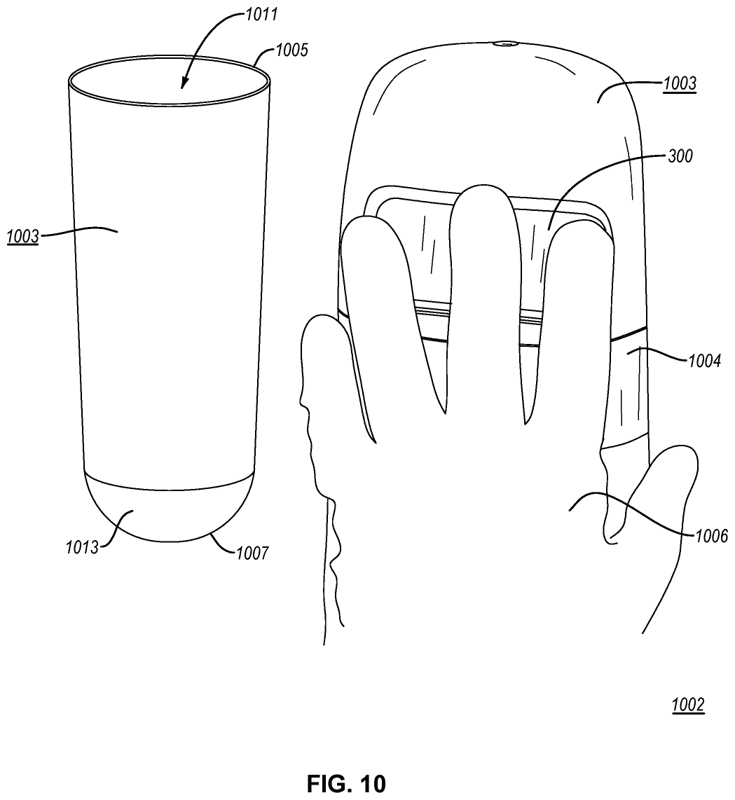

FIGS. 10-18 collectively illustrate a method of making an adjustable prosthetic socket in accordance with another embodiment of the invention.

Referring to FIGS. 10-18, they illustrate a method of making an adjustable member of FIG. 1. The method includes heating the panel 300 to a molding or shaping temperature, e.g., with a heat gun, oven, or other heating source (step 1002). The molding temperature can be in the range from about 120° F. to about 305° F. In a preferred embodiment, the heated and moldable panel 300 is pressed onto the surface of a flexible inner pre-socket 1003 such that the heated panel is molded to a curved shape of the flexible inner-pre socket after being cooled. Panel 300 is temporarily secured to the flexible inner socket 115 with a securing mechanism 1004, e.g., tape, band, string, hook and loop strap, combinations of the same, and the like. Next, not shown, a stockinet, liner, and/or sock is arranged over the adjustable member 300 and flexible inner socket 1003.

The flexible inner pre-socket is described and utilized to form a flexible inner socket as described in U.S. patent application Ser. Nos. 16/897,425 and 17/901,375, each of which are hereby incorporated by reference as if fully set forth herein. The flexible inner pre-socket has not been molded into a flexible inner socket but is a precursor product with no contours resembling the contours of the residual limb. It should be understood that any conventional liner can be used instead of the flexible inner pre-socket 1003.

In one embodiment, the flexible inner pre-socket can be formed with blow molding, injection material, or other techniques. In one embodiment, the flexible inner pre-socket or socket is formed from a thermoformable material. The thermoformable material includes one or more of a thermoplastic polyurethane (T.P.U.) material, a thermoplastic polyurethane foam material, a thermoplastic vulcanizate (TPV) material, thermoplastic elastomer (TPE) materials, a thermoset material, a rubber material, an ultra-low density polyethylene (ULDPE) material, an ethylene vinyl acetate (E.V.A.) material, a styrene material, and blends of the same.

The flexible inner pre-socket 1003 has a first end 1005, a second end 1007, and the first end 1005 has an opening 1011 configured and dimensioned to receive at least a portion of a residual limb of a user. The second end 1007 is a closed end 1013 in a rounded type orientation. Optionally and/or alternatively, the second end has a hole or channel that extends through the thickness of the flexible inner pre-socket 1003. A cavity or partial channel is formed extending from the open first end 1005 to the closed second end 1007. The closed end 1013 can have any type of geometry, e.g., square, rectangle, cone, etc. The flexible inner pre-socket has an outer circumference that is smaller than the inner circumference of the conical cup 102 of the adjustable prosthetic socket 100.

The flexible inner pre-socket 1004 includes a thermoplastic or thermoformable material as described herein such that when it is heated, it becomes pliable and stretchable so as to assume a new shape when formed and thereby holding that shape when cooled. In one embodiment, the heating temperature is in a range from about 170° F. to about 350° F., and the temperature when cooled is in a range from about 125° F. or below. In a preferred embodiment, the heating temperature is in a range from about 225° F. to about 280° F. (and when cooled to about 150° F. or below). In a most preferred embodiment, the heating temperature is in a range from about 300° F. to about 315° F.

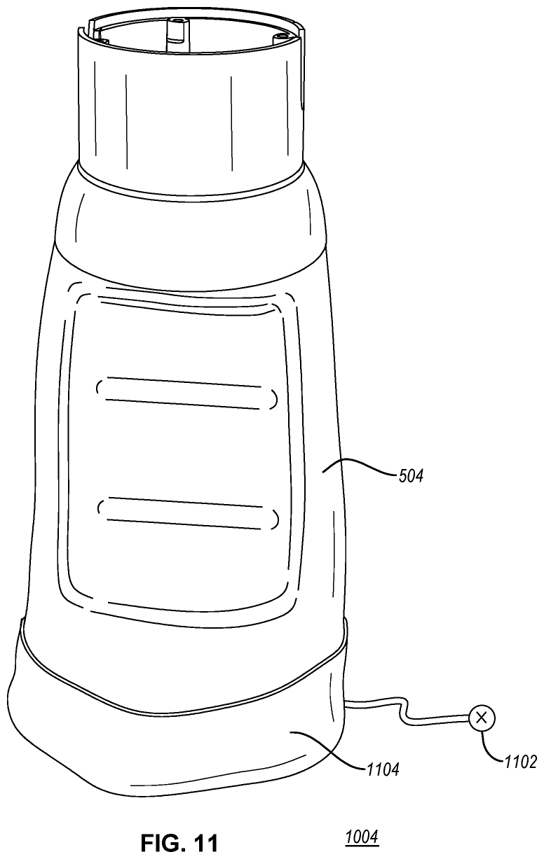

Referring to FIG. 11, the flexible inner pre-socket with the adjustable member 300 attached (formed from step 1002) is arranged into the conical cup of a heat moldable prosthetic socket that has not yet been heated and does not have any contours of the residual limb but is a blank shell with a conical cup and lower portion as described with reference to U.S. patent application Ser. No. 17/901,375, which is hereby incorporated by reference as if fully set forth herein.

The entire assembly is heated with an oven or other heat source to a shaping temperature in a range from about 120° F. to about 300° F. In one embodiment, the oven can be the oven described with reference to U.S. patent application Ser. No. 17/511,22, which is hereby incorporated by reference as if fully set forth herein. The entire assembly includes a flexible inner pre-socket with an adjustable member 300 arranged in the conical cup of a heat moldable prosthetic socket. The heated assembly is pulled and arranged over a mold of a residual limb or directly on a residual limb with heat resistant gloves in step 1090. The positive mold has an outer surface that matches or mimics the surface and contours of a residual limb of a patient. In a preferred embodiment, the positive model has been globally reduced as is known in the art.

A vacuum source 1102 is utilized to add a compressive force to compress the conical cup 102, adjustable member 300, and flexible inner socket 115 onto the surface of the residual limb or a mold or model of a residual limb, thereby allowing the assembly to conform to contours of the residual limb. The vacuum force is a compressive force that is utilized by using a vacuum sleeve 1104. In operation, sleeve 1104 is rolled up, and a vacuum is applied until the heated flexible socket and prosthetic socket are cooled. Optionally, a stockinette is pulled over the flexible inner pre-socket, and the vacuum is applied through a vacuum hose. This process takes about one minute or more, and cooling takes a few a bit longer. Of course, the process may use other compressive mechanisms, such as hands to compress the heated conical cup, or other external forces and combinations of the same and like.

During the process, the heated assembly is molded to conform to one or more contours of the model or the residual limb to form a recess 112, flexible inner socket 115, where the conical cup and flexible inner socket mimic one or more contours of the residual limb. The shaping or molding time can be in the range of five minutes to fifteen minutes or any sub-range within. During the molding, portions of the assembly, e.g., the conical cup, are stretched circumferentially directly over the model of the residual limb so that the conical cup is shaped to fit the residual limb by mimicking one or more contours of the model. Various techniques may be used to mold the prosthetic socket to the model, e.g., hand shaping with gloves, vacuum forming, applying a compression sleeve or wrap, and the like.

When cooled to room temperature, the assembly is hardened and removed from the model or residual limb. After this process, the adjustable member or panel 300 resides in a recess 112 formed in a sidewall of the conical cup 102 (FIG. 3). Optionally and/or alternatively, the prosthetic socket, including the recess and the flexible inner socket, is optionally trimmed, ground and/or buffed to remove a predetermined amount of material and into a desired geometry. Optionally, the edges of either the flexible inner socket and prosthetic socket can also be heated with a heat gun and flared and shaped as desired.

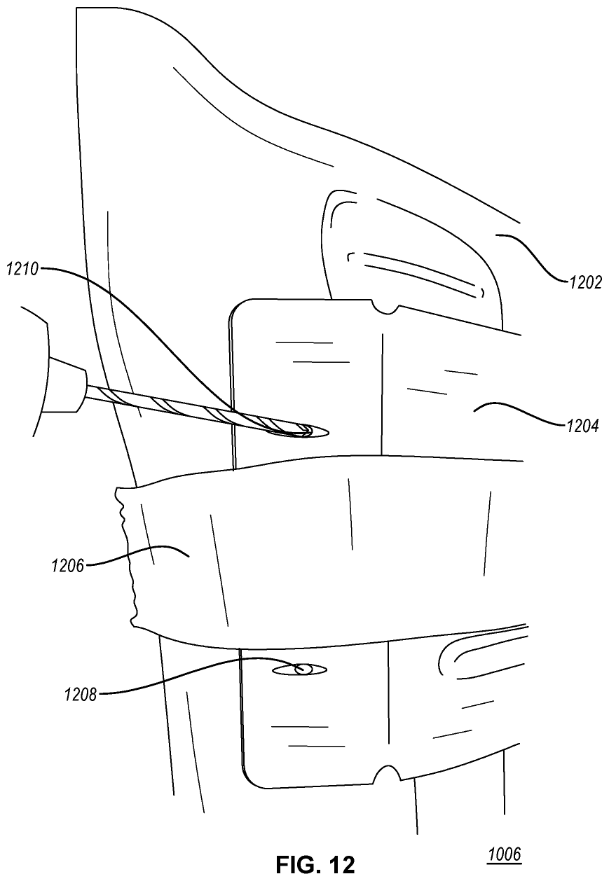

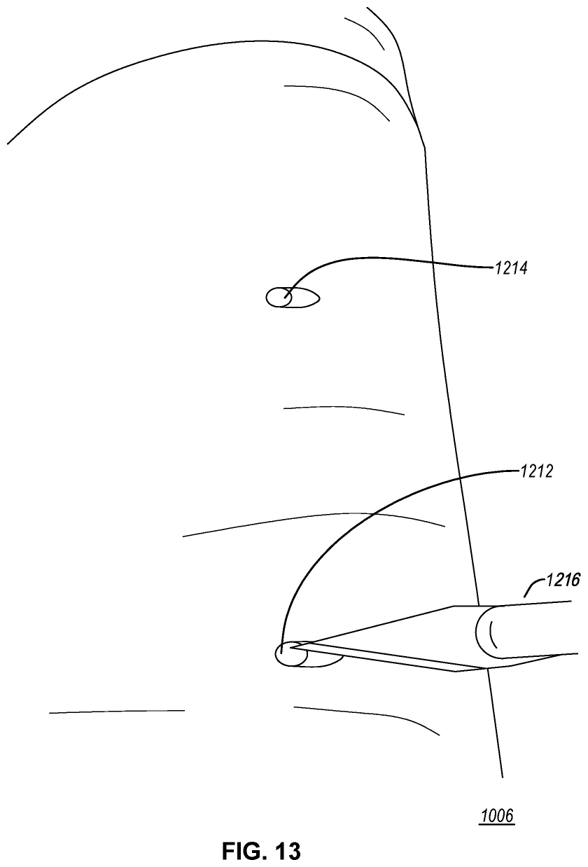

Referring now to FIGS. 12-13, in step 1006, holes are formed in the outer surface of the molded prosthetic socket 1202 to provide access to the adjustable panel. That is, the holes will correspond to exits and entrances for the wire. In a preferred embodiment, a drill template 1204, including alignment holes corresponding to each drill site, is temporarily attached to the socket 1202 by tape 1206 or another attachment mechanism, e.g., strap, hook and loop strap, wire, glue combinations of the same, and the like. The template 1204 is used to drill holes 1208, 1210, 1212 and 1214. The holes can be cleaned out, as shown in FIG. 13, with a cleaning device 1216, e.g., a sharp object, a knife, a cleaning drill bit, sandpaper, and the like. The drill holes 1208, 1210, 1212, and 1214 go completely through the sidewall of the socket 1202.

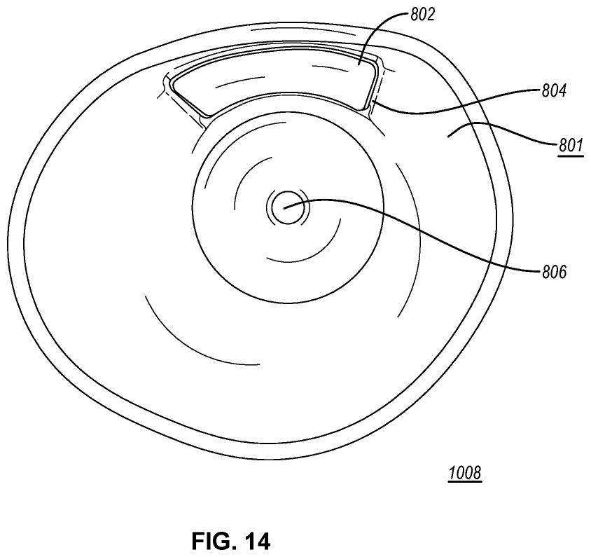

Referring to FIG. 14, a top down view of the prosthetic socket includes an adjustable panel 802 formed in a recess 804 of an inner wall 801 of the socket. The socket includes an opening at a proximal end to receive a residual limb and a small opening at the distal end 806. The sidewalls of the socket 801 do not include any openings, and any input holes or output holes for receiving the tensioning line have not yet been formed.

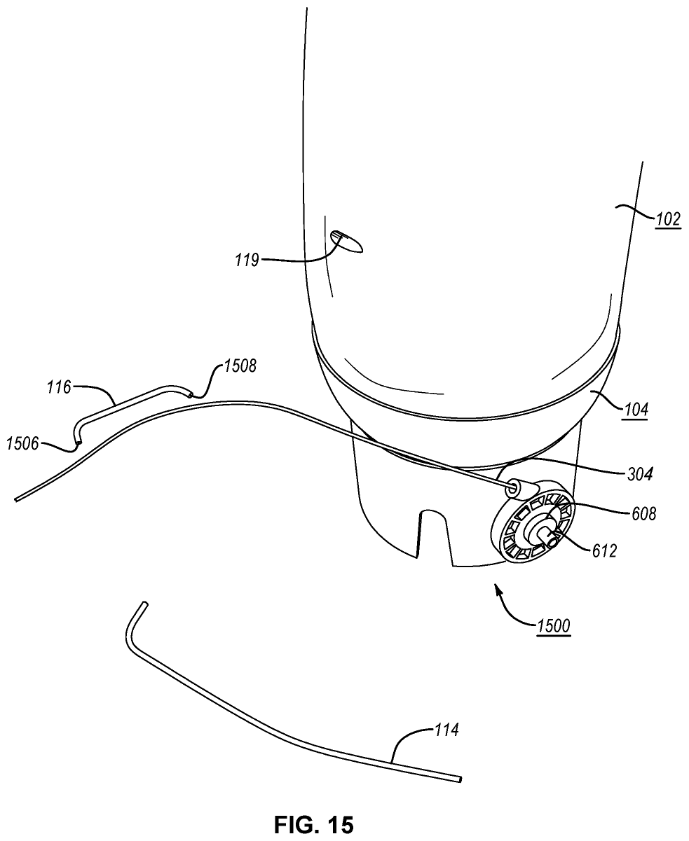

Referring to FIG. 15, components of an actuator 108 in a disassembled state are shown. In this step, 1500, the actuator 108, cable 304, first wire guide 114, and second wire guide 116 are assembled, and the panel 300 is also installed. The cable 304 is coupled or affixed to the sprocket 608 and arranged through the alignment tubing 114 and into the socket hole 119. Next, cable 304 is passed through passageway 302 and out a socket hole 118. Next, it passed through alignment tubing 116 via hole 1506 and out of hole 1508. Next, it passes into the socket through hole 120, through an upper passageway 306, and to a termination point 308. In this embodiment, the termination point 308 can include a plurality of holes in the socket in order to knot the cable at an exit. Referring to FIGS. 3, 4, and 10, the panel is now adjustable by rotating the actuator 108 as described herein.

In one embodiment, components herein may be provided as a kit. The kit can include one or more tensioning lines 304, a tightening mechanism 108, one or more alignment guides 116, 114, and an alignment template 1204 and instructions for use. The kit can also be used for a flexible inner pre-socket or conventional liner and a heat moldable prosthetic socket. Of course, other components as described herein may also be used included.

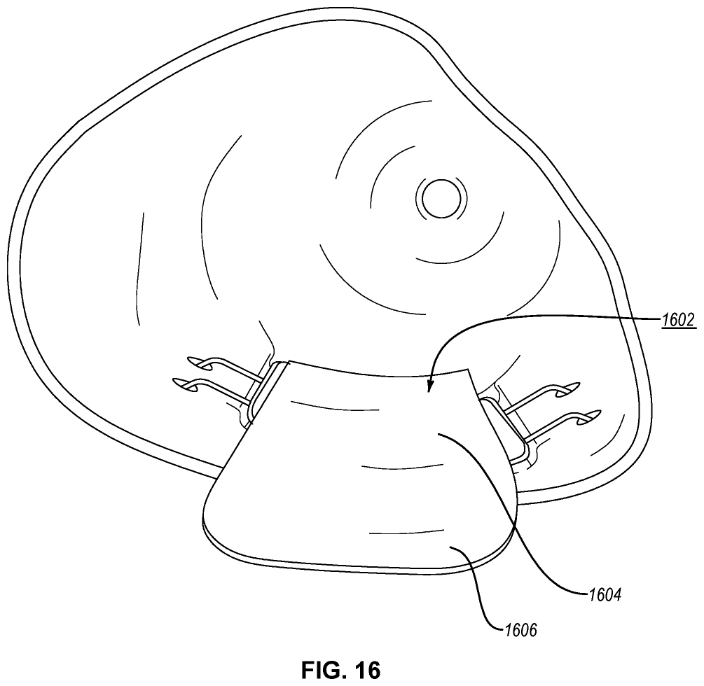

Referring to FIG. 16, optionally and/or alternatively, a panel 1602, e.g., an adhesive panel, is attached to panel 300. The panel 1602 can include any flexible material, e.g., leather, synthetic leather, or blended material. In a preferred embodiment, one side of the material includes an adhesive portion on one or more portions of the panel, e.g., a first portion 1604 and a second portion 1606. The first portion 1604 is attached to at least a portion of the panel 300 (FIG. 11), and second end portion 1606 is attached to the socket (FIG. 12). Next, the formed and trimmed flexible liner socket 115 is placed in the socket as shown in FIG. 1.

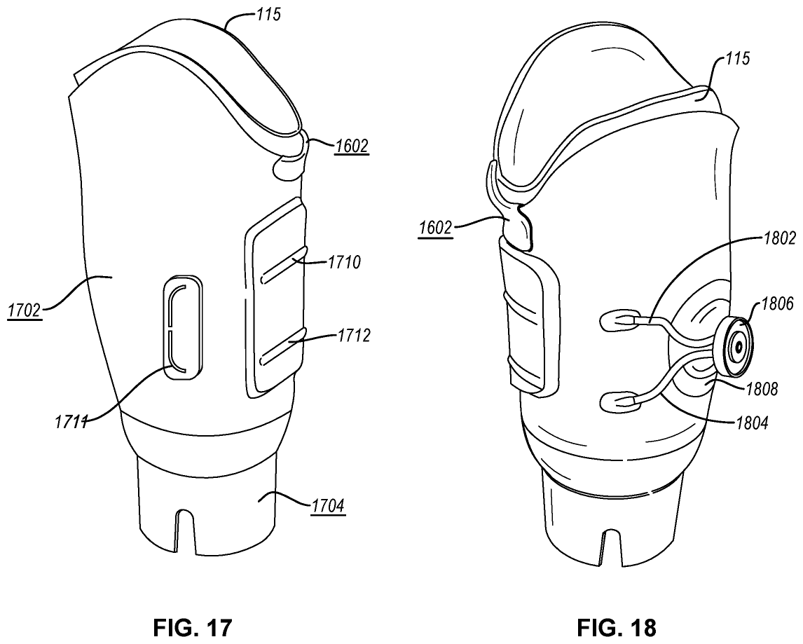

FIG. 17 illustrates a left backside perspective view of an adjustable prosthetic socket in accordance with another embodiment of the invention. FIG. 18 illustrates a right backside perspective view of an adjustable prosthetic socket in accordance with another embodiment of the invention.

Referring to FIGS. 17-18, an adjustable prosthetic socket is shown with reference to number 1700. The adjustable socket 1700 includes a flexible inner socket 115 as described herein or a conventional liner. The adjustable socket 1700 includes a conical cup 1702 and lower portion 1704 as described herein. The sidewall of the socket 1700 includes channels or partial channels 1712 and 1710 and configured to receive one or more cables, wires, or other linkage from a tightening mechanism 1806. In addition, an alignment tube 1711 is also utilized, which is similar to the alignment tube 116. The socket also includes a recessed panel as described herein. In this embodiment, the socket includes a BOA® tightening mechanism. These BOA® tightening mechanisms are described with reference to U.S. Pat. Nos. 8,468,657; 9,138,030; 9,408,437; 9,770,070; 10,772,388; 10,888,139; 10,952,505; 11,452,342; 11,457,698; and 11,633,020, each of which is hereby incorporated by reference as if fully set forth herein. Of course, other tensioning mechanisms may also be utilized along with other locations, e.g., conical cup or lower portion. In this embodiment, the tensioning mechanism 1806 is attached with a skirt 1808 and an attachment mechanism, e.g., adhesive, screws, rivet, and other mechanical mechanisms and the like. A tensioning line, e.g., cable, is fed through alignment channels 1804 and 1802. In operation, the actuator is operated to move the panel from a first location to a second location and apply a compressive force to a residual limb.

The inventions and methods described herein can be viewed as a whole or as a number of separate inventions that can be used independently or mixed and matched as desired. All inventions, steps, processes, devices, and methods described herein can be mixed and matched as desired. All previously described features, functions, or inventions described herein or by reference may be mixed and matched as desired. It will be apparent to those skilled in the art that various modifications and variations can be made in the present invention without departing from the spirit or scope of the invention. Thus, it is intended that the present invention cover the modifications and variations of this invention, provided they come within the scope of the appended claims and their equivalents.

1. An adjustable prosthetic socket, comprising:
a heat moldable socket comprising a proximal open end, a distal end that is substantially closed, and a wall surrounding and extending from the distal end to the proximal open end, the heat moldable socket configured to at least partially surround and secure a residual limb;
at least one recess in the wall of the heat moldable socket, wherein the at least one recess has no opening or cutouts;
at least one adjustable panel sized to be arranged in the at least one recess, wherein the at least one adjustable panel resides entirely inside the heat moldable socket and does not extend outside of the heat moldable socket; and
an actuator coupled to a cable or wire configured to change an inner circumference of the heat moldable socket by operation of the actuator applying tensions to the cable or wire and moving the at least one adjustable panel from a first position to a second position, wherein the second position reduces at least a portion of the inner circumference of the heat moldable socket.

2. The adjustable prosthetic socket of claim 1, wherein the actuator is configured to apply tension to the cable or wire.

3. The adjustable prosthetic socket of claim 1, further comprising a flexible inner socket configured to be arranged in a portion of the socket.

4. The adjustable prosthetic socket of claim 1, wherein the heat moldable socket comprises an injection molded premade thermoplastic elastomer socket comprising a thermoformable material.

5. The adjustable prosthetic socket of claim 4, wherein the injection molded premade thermoplastic elastomer socket is configured to be heated to a shaping temperature and becomes formable and stretchable.

6. The adjustable prosthetic socket of claim 1, wherein the at least one adjustable panel comprises one of a thermoplastic plastic and polymeric material.

7. The adjustable prosthetic socket of claim 1, wherein the at least one adjustable panel is configured to be heated to a formable temperature so that the at least one adjustable panel can be molded into a predetermined geometric configuration.

8. The adjustable prosthetic socket of claim 1, wherein the actuator cannot rotate more than one of 360 degrees, 180 degrees, and 90 degrees.

9. An adjustable prosthetic socket, comprising:
a pre-made socket comprising a proximal open end, a distal end that is a substantially closed end, and a receptacle extending from the distal end to the proximal open end, the pre-made socket configured to at least partially surround and secure a residual limb;
at least one recess in a wall of the pre-made socket; and
at least one adjustable panel having a dimension that allows it to be arranged in the at least one recess, wherein no portion of the at least one adjustable panel extends outside an interior region of the pre-made socket;
a tightening mechanism comprising a base rotationally coupled to a dial, wherein the tightening mechanism is configured to:
couple to a tensioning line; and
rotate no more than 360 degrees.

10. The adjustable prosthetic socket of claim 9, further comprising a flexible inner socket.

11. The adjustable prosthetic socket of claim 9, wherein the at least one adjustable panel comprises one or more alignment features configured to assist with guiding the tensioning line.

12. The adjustable prosthetic socket of claim 9, wherein the tensioning line comprises one or more of a wire, a cable, a lace, a woven material, combinations of the same and the like.

13. The adjustable prosthetic socket of claim 12, wherein the tensioning line comprises a material selected from the group consisting of a metal material, a polyester material, an alloy material, a synthetic material, combinations of the same and the like.

14. A heat moldable adjustable prosthetic socket, comprising:
a heat moldable socket comprising a proximal open end, a distal end that is substantially closed, and a wall extending from the distal end to the proximal open end, the heat moldable adjustable prosthetic socket configured to at least partially surround and secure a residual limb;
a recess in the wall of the heat moldable adjustable prosthetic socket;
a panel configured to be arranged in the recess; and
an actuator coupled to a tensioning line that is configured to change an inner circumference of the heat moldable adjustable prosthetic socket by moving the panel from a first position to a second position by operation of the actuator, wherein the second position reduces an inner circumference of the socket.

15. The heat moldable adjustable prosthetic socket of claim 14, wherein the panel comprises one of a thermoplastic plastic material and a polymeric material.

16. The heat moldable adjustable prosthetic socket of claim 15, wherein the heat moldable socket has a shaping temperature in a range of about 120° F. to about 305° F. and any sub-range thereof.

17. The heat moldable adjustable prosthetic socket of claim 16, wherein the panel is heat moldable.

18. The heat moldable adjustable prosthetic socket of claim 14, further comprising a flexible inner socket.

19. The heat moldable adjustable prosthetic socket of claim 14, further comprising a liner.

20. The heat moldable adjustable prosthetic socket of claim 14, wherein the panel comprises a plurality of alignment features.

21-30. (canceled)
